(19) United States
(12) Patent Application Publication
(10) Pub. No.: US 2025/0361248 A1
(43) Pub. Date: Nov. 27, 2025

(54) PROTAC COMPOUND WITH CYCLOPHILIN A DEGRADATION ACTIVITY, PREPARATION METHOD THEREFOR, AND USE THEREOF

(71) Applicant: SHENZHEN BAY LABORATORY, Shenzhen (CN)

(72) Inventors: Wenjun LIU, Shenzhen (CN); Lei SUN, Shenzhen (CN); Heqiao LI, Shenzhen (CN); Wenxian YANG, Shenzhen (CN); Xiaoyuan BAI, Shenzhen (CN); He ZHANG, Shenzhen (CN)

(21) Appl. No.: 19/103,028

(22) PCT Filed: Feb. 28, 2023
(86) PCT No.: PCT/CN2023/078658

§ 371 Date: Feb. 11, 2025

(30) Foreign Application Priority Data
Aug. 11, 2022 (CN) 202210957971.X

(51) Int. Cl. C07F 7/08; A61K 31/695; A61P 11/00

(52) U.S. Cl. CPC C07F 7/0836; A61K 31/695; A61P 11/00

(57) ABSTRACT

Disclosed are a PROTAC compound with cyclophilin A degradation activity, a preparation method therefor, and use thereof. The structural formula of the PROTAC compound is represented by Formula (I). The compound represented by Formula (I) provided by the present invention can be used for preventing and/or treating CypA-mediated diseases, such as CypA-mediated inflammation, autoimmune diseases, and/or tumors. The present invention also provides a pharmaceutical composition comprising the compound represented by Formula I as an active ingredient and at least one pharmaceutically acceptable carrier, excipient, and/or diluent. The PROTAC compound provided by the present invention can degrade CypA protein in a targeted manner, so that the PROTAC compound can be used for preparing medicaments for treating inflammation, autoimmune diseases, tumors, and other related diseases. The compound represented by Formula I in the present invention has a significant inhibitory function on virus-induced pneumonia, rheumatoid arthritis, and lung cancer cell migration and infiltration.

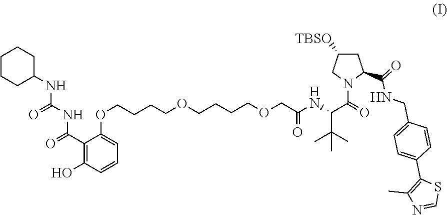 [SMILES: Cc1ncsc1-c1ccc(CNC(=O)[C@@H]2C[C@@H](O[Si](C)(C)C(C)(C)C)CN2C(=O)[C@@H](NC(=O)COCCCCOCCCCOc2cccc(O)c2C(=O)NC(=O)NC2CCCCC2)C(C)(C)C)cc1]

### RELATED APPLICATIONS

The present application is a U.S. National Phase of International Application Number PCT/CN2023/078658 filed on Feb. 28, 2023, which claims priority to Chinese Application Number 202210957971.X filed on Aug. 11, 2022.

### TECHNICAL FIELD

The present invention relates to a PROTAC compound with cyclophilin A degradation activity, a preparation method therefor, and use thereof, which belongs to the field of biomedicine.

### BACKGROUND ART

Cyclophilin A (CypA) is a multifunctional immunomodulatory protein widely expressed in eukaryotic cells. During the inflammatory process caused by viral infection, CypA can promote the production of inflammatory cytokines by upregulating the NF-κB signaling pathway. During the development of autoimmune diseases such as rheumatoid arthritis, systemic lupus erythematosus, and psoriasis, CypA can promote leukocyte migration and induce the expression of chemokines and cytokines. The expression of CypA is significantly increased in various tumor-related diseases, which is closely related to the occurrence, metastasis, and prognosis of tumors. CypA is therefore an important target protein for treating inflammation, autoimmune diseases, and tumors.

PROTAC (proteolysis-targeting chimera) consists of three moieties: a small-molecule ligand that recognizes the target protein, a linker, and an E3 ubiquitin ligase ligand, and can directly degrade the target protein, so as to achieve the effect of treating related diseases. At present, there are no reports on CypA-targeted PROTAC drugs.

### SUMMARY OF THE INVENTION

It is an object of the present invention to provide a PROTAC compound capable of achieving CypA degradation. The PROTAC compound can degrade CypA in a targeted manner and can be used for treating CypA-mediated inflammation, autoimmune diseases, tumors, and other related diseases.

The structural formula of the PROTAC compound provided by the present invention is represented by Formula I:

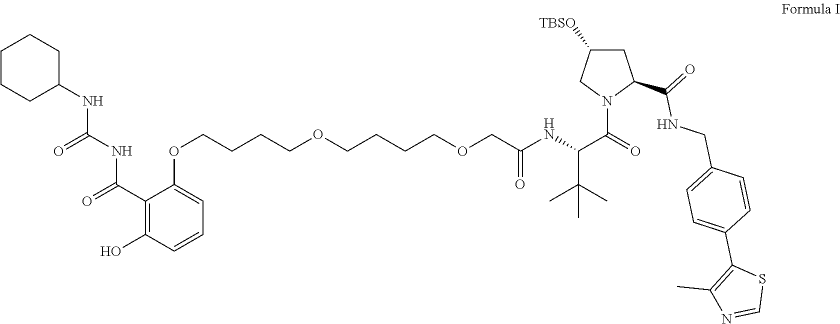

in Formula I, TBSO represents tert-butyldimethylsiloxy.

The present invention further provides a preparation method for the compound represented by Formula I, comprising the steps of:
S1, subjecting a compound represented by Formula 1 to a hydrolysis reaction in the presence of a basic compound I and benzyl alcohol to obtain a compound represented by Formula 2;

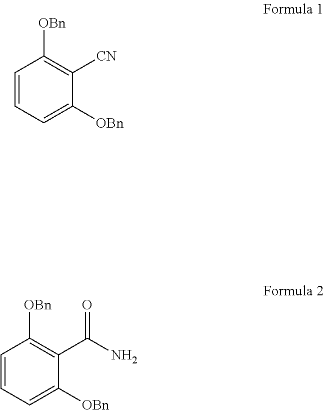

wherein Bn represents benzyl;
S2, subjecting the compound represented by Formula 2 to a nucleophilic addition reaction with isocyanatocyclohexane to obtain a compound represented by Formula 3;

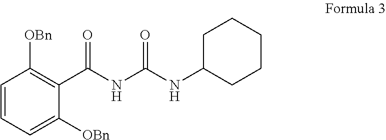 [SMILES: O=C(NC(=O)c1c(OCc2ccccc2)cccc1OCc1ccccc1)NC1CCCCC1]

S3, reducing the compound represented by Formula 3 with palladium on carbon to obtain a compound represented by Formula 4;

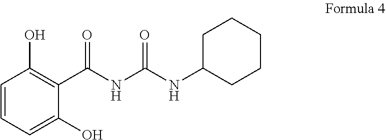 [SMILES: O=C(NC(=O)c1c(O)cccc1O)NC1CCCCC1]

S4, subjecting ((4-bromobutoxy)methyl)benzene to a nucleophilic substitution reaction with butane-1,4-diol in the to obtain presence of potassium hydroxide 4-(4-(benzyloxy)butoxy)butan-1-ol;
S5, subjecting the 4-(4-(benzyloxy)butoxy)butan-1-ol to a nucleophilic substitution reaction with tert-butyl 2-bromoacetate in the presence of tetrabutylammonium bromide and a basic compound II to obtain a compound represented by Formula 5;

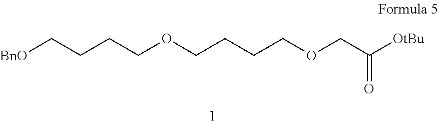 [SMILES: CC(C)(C)OC(=O)COCCCCOCCCCOCc1ccccc1]

in Formula 5, Bn represents benzyl, and tBU represents tert-butyl;
S6, reducing the compound represented by Formula 5 with palladium on carbon to obtain a compound represented by Formula 6;

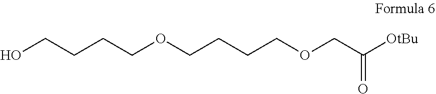 [SMILES: CC(C)(C)OC(=O)COCCCCOCCCCO]

S7, subjecting the compound represented by Formula 6 to a Mitsunobu reaction with the compound represented by Formula 4 in the presence of triphenylphosphine and diisopropyl azodicarboxylate to obtain a compound represented by Formula 7;

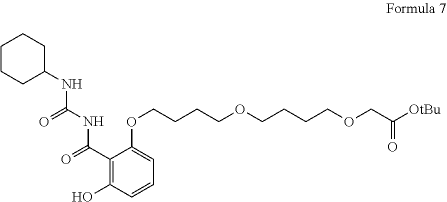 [SMILES: CC(C)(C)OC(=O)COCCCCOCCCCOc1cccc(O)c1C(=O)NC(=O)NC1CCCCC1]

S8, subjecting the compound represented by Formula 7 to a deprotection reaction in the presence of trifluoroacetic acid to obtain a compound represented by Formula 8;

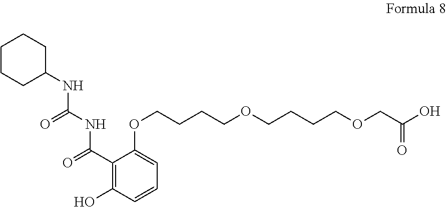 [SMILES: O=C(O)COCCCCOCCCCOc1cccc(O)c1C(=O)NC(=O)NC1CCCCC1]

S9, subjecting a compound represented by Formula 9 to a nucleophilic substitution reaction with tert-butyldimethylsilyl chloride in the presence of N,N-diisopropylethyl amine to obtain a compound represented by Formula 10;

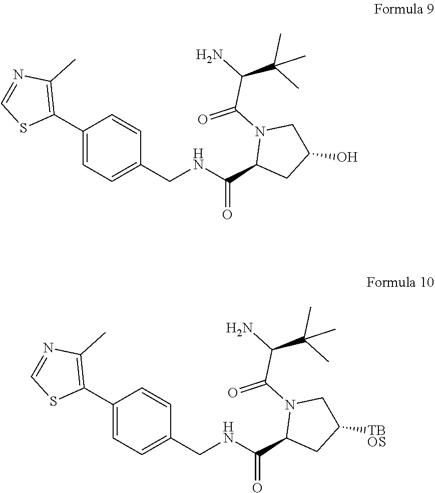

in Formula 10, TBSO represents tert-butyldimethylsiloxy; and

S10, subjecting the compound represented by Formula 8 to a condensation reaction with the compound represented by Formula 10 in the presence of 1-ethyl-(3-dimethylaminopropyl)carbodiimide hydrochloride, 1-hydroxybenzotriazole, and N,N-diisopropylethyl amine to obtain the compound represented by Formula I.

In the above preparation method, in step S1, the basic compound I is KOH (potassium hydroxide);
the molar ratio of the compound represented by Formula 1 to the benzyl alcohol is 1:(5 to 15), preferably 1:10;
the temperature for the hydrolysis reaction is 120 to 150° C., and the time is 8 to 16 h;
the hydrolysis reaction is carried out in water;
in step S2, the nucleophilic addition reaction is carried out under reflux for 12 to 24 h;
in step S3, the mass percentage content of palladium in the palladium on carbon is 10%;
the reduction reaction is carried out in ethanol.

In the above preparation method, in step S4, the basic compound II is KOH;
the molar ratio of the ((4-bromobutoxy)methyl)benzene to the butane-1,4-diol is 1:(2 to 5), preferably 1:3;
the temperature for the nucleophilic substitution reaction is 20 to 40° C., and the time is 1 to 5 h; in step S5, the basic compound III is NaOH;
the molar ratio of the 4-(4-(benzyloxy)butoxy)butan-1-ol to the tert-butyl 2-bromoacetate is 1:(1 to 5), preferably 1:2;
the temperature for the nucleophilic substitution reaction is 20 to 40° C., and the time is 12 to 24 h.

In the above preparation method, in step S7, the molar ratio of the compound represented by Formula 6 to the compound represented by Formula 4 is 1:(1 to 1.5), preferably 1:1;
the molar ratio of the compound represented by Formula 6, the triphenylphosphine, and the diisopropyl azodicarboxylate is 1:(1 to 2):(1 to 2), preferably 1:1.5:1.5;
the temperature for the condensation reaction is 0 to 25° C., and the time is 1 to 5 h;
in step S8, the temperature for the deprotection reaction is 20 to 40° C., and the time is 1 to 5 h;
in step S9, the molar ratio of the compound represented by Formula 9 to tert-butyldimethylsilyl chloride is 1:(1 to 5), preferably 1:2;
the molar ratio of the compound represented by Formula 9 to the N,N-diisopropylethyl amine is 1:(5 to 15), preferably 1:10;
the temperature for the substitution reaction is 0 to 25° C., and the time is 12 to 18 h;
in step S10, the molar ratio of the compound represented by Formula 8 to the compound represented by Formula 10 is 1:(1 to 1.5), preferably 1:1.1;
the molar ratio of the compound represented by Formula 8, the 1-ethyl-(3-dimethylaminopropyl)carbodiimide hydrochloride, the 1-hydroxy benzotriazole, and the N,N-diisopropylethyl amine is 1:(1 to 2):(1 to 2):(1 to 2), preferably 1:1.5:1.5:1.5;
the temperature for the condensation reaction is 0 to 25° C., and the time is 12 to 18 h.

The compound represented by Formula I provided by the present invention can be used for preventing and/or treating CypA-mediated diseases, such as CypA-mediated inflammation, autoimmune diseases, and/or tumors;
preferably, the inflammation comprises pneumonia;
the autoimmune diseases comprise rheumatoid arthritis, systemic lupus erythematosus, and psoriasis;
the tumors comprise lung tumors.

Also within the scope of the present invention is the use of the compound represented by Formula I for preparing a product having any of the following functions:
1) degrading CypA protein;
2) alleviating influenza virus-induced pneumonia;
3) treating rheumatoid arthritis;
4) inhibiting the migration and infiltration of cancer cells.

The present invention also provides a method for treating CypA-mediated diseases in a subject, the method comprising administering to the subject a therapeutically effective amount of the compound represented by Formula I.

The present invention also provides a pharmaceutical composition comprising the compound represented by Formula I as an active ingredient and at least one pharmaceutically acceptable carrier, excipient, and/or diluent.

### BRIEF DESCRIPTION OF THE DRAWINGS

FIG. 1 shows a synthetic scheme for the compound represented by Formula I in the present invention.

FIG. 2 shows the effect of the compound represented by Formula I on cell activity.

FIGS. 3A and 3B shows the results of the degradation activity of the compound represented by Formula I on CypA, where panel A shows the results of the degradation of CypA by the compound represented by Formula I at different concentrations, and FIG. 3B shows the grayscale analysis of the relative expression of CypA of FIG. 3A in FIGS. 3A and 3B.

FIGS. 4A-4H shows the results of the alleviation of influenza B virus-induced pneumonia by the compound represented by Formula I, where FIG. 4A shows the detection of the expression of CypA in mice of each group, FIG. 4B shows the lung index of mice in each group, FIG. 4C shows the pathology of mice in each group, FIG. 4D shows the pathological scores of mice in each group, FIG. 4E shows the relative expression of Illb mRNA in mice of each group, FIG. 4F shows the relative expression of Il6 mRNA in mice of each group, FIG. 4G shows the relative expression of Tnfa mRNA in mice of each group, and FIG. 4H shows the relative expression of Ifng mRNA in mice of each group.

FIGS. 5A-5D shows the results of the inhibition of rheumatoid arthritis by the compound represented by Formula I, where FIG. 5A shows the HE images of the ankles of rats in each group, FIG. 5B shows the joint swelling rate of rats in each group, FIG. 5C shows the joint index of rats in each group, and FIG. 5D shows the ankle pathological scores of rats in each group.

FIGS. 6A and 6B shows the results of the inhibition of the migration and infiltration of lung cancer cells by the compound represented by Formula I, where FIG6A shows the results of the observation of the migration and infiltration of cells in each group by the Traswell method, and FIG. 6B shows the cell counts of FIG. 6A in FIGS. 6A and 6B.

### DETAILED DESCRIPTION OF THE INVENTION

The experimental methods used in the following examples are conventional, unless otherwise specified.

The materials, reagents, etc. used in the following examples are all commercially available, unless otherwise specified.

### Example 1. Design and Synthesis of a Compound of Formula I for Degrading CypA in a Targeted Manner

The synthetic scheme is shown in FIG. 1.

### (1) Synthesis of 2,6-Bis(Benzyloxy)Benzamide (Compound 2)

Compound 1 (2.0 g, 6.34 mmol) and potassium hydroxide (2.85 g, 50.73 mmol) were dissolved in 20 mL of benzyl alcohol and 2 mL of water. The mixture was heated to 130°° C. under a microwave, reacted for 12 h, and then concentrated in vacuo to remove the solvent. Water (200 mL) was added to the residue. The filtered solid was purified by silica gel flash column chromatography and eluted with (petroleum ether/ethyl acetate=20:1 to 1:1) to obtain compound 2 (1.2 g).

¹H NMR (400 MHz, DMSO) δ 7.63 (s, 1H), 7.45 (d, J=7.1 Hz, 4H), 7.38 (t, J=7.3 Hz, 4H), 7.34-7.27 (m, 3H), 7.21 (t, J=8.3 Hz, 1H), 6.72 (d, J=8.4 Hz, 2H), 5.11 (s, 4H).

### (2) Synthesis of 2,6-Bis(Benzyloxy)-N-(Cyclohexylcarbamoyl)Benzamide (Compound 3)

Compound 2 (1.2 g, 3.6 mmol) and isocyanatocyclohexane (0.54 g, 4.32 mmol) were dissolved in toluene (30 ml), refluxed for 18 hours, and concentrated in vacuo to obtain a crude product. The crude product was purified by silica gel flash column chromatography and eluted with a mixture of ethyl acetate/petroleum ether (1:3, v/v) to obtain compound 3 (1.1 g) as a white solid.

¹H NMR (400 MHz, CDCl₃) δ 8.39 (d, J=7.7 Hz, 1H), 7.80 (s, 1H), 7.38-7.20 (m, 11H), 6.58 (d, J=8.5 Hz, 2H), 5.13 (s, 4H), 3.75 (dd, J=7.9, 3.8 Hz, 1H), 1.97 (d, J=10.2 Hz, 2H), 1.74 (dd, J=9.0, 3.9 Hz, 2H), 1.65-1.55 (m, 1H), 1.43-1.22 (m, 5H).

### (3) Synthesis of N-(Cyclohexylcarbamoyl)-2,6-Dihydroxy Benzamide (Compound 4)

Compound 3 (1.1 g, 2.4 mmol) and 10% Pd—C (0.22 g) were added to EtOH (30 ml) and stirred under a hydrogen atmosphere at 25° C. for 16 h. The mixture was filtered. The filtrate was evaporated in vacuo. The residue was purified by silica gel flash column chromatography and eluted with a mixture of ethyl acetate/petroleum ether (1:1, v/v) to obtain a white solid 4 (520 mg).

¹H NMR (400 MHz, DMSO) δ 11.68 (s, 2H), 10.53 (s, 1H), 8.23 (s, 1H), 7.24 (s, 1H), 6.43 (d, J=8.2 Hz, 2H), 3.61 (s, 1H), 1.86 (s, 2H), 1.66 (s, 2H), 1.54 (s, 1H), 1.34-1.23 (m, 5H).

### (4) Synthesis of 4-(4-(Benzyloxy)Butoxy)Butan-1-Ol

((4-Bromobutoxy)methyl)benzene (2.0 g, 8.23 mol) and butane-1,4-diol (2.22 g, 24.68 mol) were dissolved in DMSO (15 mL), and potassium hydroxide (2.31 g, 41.13 mmol) was added. The reaction mixture was stirred at 25° C. for 2 hours. Once LCMS showed the reaction was completed, water (200 ml) was poured into the reaction solution. The mixture was extracted with dichloromethane (200 ml×3). The solvent was removed in vacuo, and the resulting product was purified by silica gel column chromatography and eluted with (petroleum ether/ethyl acetate=0 to 10%) to obtain a colorless oil (1.1 g).

¹H NMR (400 MHz, CDCl₃) δ 7.35-7.25 (m, 5H), 4.50 (s, 2H), 3.63 (t, J=5.6 Hz, 2H), 3.54-3.40 (m, 6H), 2.35 (s, 1H), 1.66 (dd, J=10.0, 4.3 Hz, 8H).

### (5) Synthesis of Tert-Butyl 2-(4-(4-(Benzyloxy)Butoxy)Butoxy)Acetate (Compound 5)

4-(4-(Benzyloxy)butoxy)butan-1-ol (1.1 g, 4.36 mmol), tert-butyl 2-bromoacetate (1.7 g, 8.72 mmol), and tetrabutylammonium bromide (2.81 g, 8.72 mmol) were dissolved in DCM (10 ml), and (5M) NaOH (10 ml) was added. The reaction mixture was stirred at room temperature for 18 hours. Once LCMS showed the reaction was completed, the reaction solution was poured into water (200 ml) and extracted with DCM (200×3). The solvent was removed in vacuo, and the resulting product was purified by silica gel column chromatography and eluted with (petroleum ether/ethyl acetate=0-20%) to obtain compound 5 as a colorless oil (502 mg).

### (6) Synthesis of Tert-Butyl 2-(4-(4-HydroxyButoxy)Butoxy)Acetate (Compound 6)

Compound 5 (3.8 g, 10.3 mmol) and 10% Pd/C (400 mg) were added to ethanol (60 ml) and stirred under a hydrogen atmosphere at 25° C. for 16 h. The mixture was filtered. The filtrate was evaporated in vacuo. The residue was purified by silica gel flash column chromatography and eluted with a mixture of ethyl acetate/petroleum ether (1:1, v/v) to obtain compound 6 (1.6 g) as a colorless oil.

1H NMR (400 MHz, CDCl3) δ 3.94 (s, 2H), 3.63 (t, J=5.8 Hz, 2H), 3.53 (t, J=6.0 Hz, 2H), 3.49-3.44 (m, 4H), 1.70-1.64 (m, 8H), 1.47 (d, J=10.4 Hz, 9H).

### (7) Synthesis of Tert-Butyl 2-(4-(4-(2-((Cyclohexylcarbamoyl)Carbamoyl)-3-Hydroxyphenoxy)Butoxy)Butoxy)Acetate (Compound 7)

Compound 6 (500 mg, 1.81 mmol), compound 4 (500 mg, 1.81 mmol), and triphenylphosphine (710 mg, 2.72 mmol) were dissolved in N,N-dimethylformamide (10 mL), cooled to 0° C., and diisopropyl azodicarboxylate (545 mg, 2.72 mmol) was added in the presence of protective nitrogen. The reaction mixture was stirred in the presence of protective nitrogen at 25° C. for 2 hours. Water was added to the reaction solution, and the mixture was extracted with dichloromethane (100 mL×3). The organic phase was concentrated in vacuo. The crude product was purified by silica gel column chromatography and eluted with (petroleum ether/ethyl acetate=20:1 to 1:1) to obtain compound 7 (250 mg) as a colorless oil.

### (8) Synthesis of 2-(4-(4-(2-((Cyclohexylcarbamoyl)Carbamoyl)-3-Hydroxyphenoxy)Butoxy)Butoxy)Acetate

Compound 7 (250 mg, 0.47 mmol) was dissolved in a mixed solvent of TFA (2 mL) and DCM (8 mL). The reaction mixture was stirred in the presence of protective nitrogen at 25° C. for 2 hours. The mixture was concentrated in vacuo. The crude product was purified by silica gel column chromatography and eluted with (dichloromethane/methanol=100:0 to 10:1) to obtain compound 8 (180 mg) as a colorless oil.

### (9) Synthesis of (2S,4R)-1-((S)-2-Amino-3,3-Dimethylbutanoyl)-4-((Tert-Butyldimethylsilyl)Oxy)-N-(4-(4-Methylthiazol-5-Yl)Benzyl)Pyrrolidine-2-Carboxamide

Compound 9 (500 mg. 1.16 mmol) and N,N-diisopropylethyl amine (1498 mg, 11.6 mmol) were dissolved in N,N-dimethylformamide (5 mL), cooled to 0° C., and tert-butyldimethylsilyl chloride (348 mg, 2.32 mmol) was added in the presence of protective nitrogen. The reaction solution was stirred in the presence of protective nitrogen at 25° C. for 16 hours. Water was added to the reaction solution, and the mixture was extracted with dichloromethane (100 mL×3). The organic phase was concentrated in vacuo. The crude product was purified by silica gel column chromatography and eluted with (petroleum ether/ethyl acetate=20:1 to 0:100 and DCM/MeOH 100:0 to 10:1) to obtain compound 10 (200 mg) as an orange oil.

### (10) Synthesis of (2S,4R)-4-((Tert-Butyldimethylsilyl)Oxy)-1-((S)-2-(2-(4-(4-(2-((Cyclohexylcarbamoyl)Carbamoyl)-3-Hydroxyphenoxy)Butoxy))Butoxy)Acetamido)-3,3- Dimethylbutanoyl)-N-(4-(4-Methylthiazol-5-Yl) Benzyl)Pyrrolidine-2-Carboxamide

Compound 8 (180 mg, 0.375 mmol), 1-ethyl-(3-dimethylaminopropyl)carbodiimide hydrochloride (108 mg, 0.5625 mmol), 1-hydroxybenzotriazole (76 mg, 0.5625 mmol), and N,N-diisopropylethyl amine (73 mg, 0.5625 mmol) were dissolved in N,N-dimethylformamide (5 mL), cooled to 0° C., and compound 11 (225 mg. 0.4125 mmol) was added in the presence of protective nitrogen protection. The reaction mixture was stirred in the presence of protective nitrogen at 25° C. for 16 hours. The mixture was concentrated in vacuo. The crude product was purified by silica gel column chromatography and eluted with (dichloromethane/methanol=50:1 to 10:1) to obtain compound I (50 mg, purity: 84%) as a white solid.

The structural characterization data of compound I were as follows:

¹H NMR (400 MHz, MeOD) δ 8.75 (d, J=7.2 Hz, 1H), 7.30 (m, 5H), 6.47 (dd, J=8.4, 3.6 Hz, 2H), 4.61 (s, 1H), 4.49 (d, J=7.2 Hz, 2H), 4.39 (d, J=11.6 Hz, 1H), 4.24 (d, J=15.5 Hz, 1H), 4.10 (dd, J=12.6, 6.4 Hz, 2H), 3.88-3.84 (m, 1H), 3.78 (d, J=15.4 Hz, 1H), 3.74-3.64 (m, 2H), 3.60 (d, J=4.4 Hz, 1H), 3.45-3.35 (m, 6H), 2.37 (d, J=4.8 Hz, 3H), 2.14-2.05 (m, 1H), 2.03-1.93 (m, 1H), 1.85 (d, J=8.4 Hz, 4H), 1.67 (dd, J=16.4, 10.3 Hz, 4H), 1.55 (d, J=12.5 Hz, 4H), 1.33-1.17 (m, 6H), 0.91 (d, J=4.8 Hz, 9H), 0.75 (s, 9H),-0.00 (d, J=3.3 Hz, 6H).

### Example 2. Assay for the Effect of the Compound Represented by Formula I on A549 Cell Activity by CCK-8 Method

I. Treatment of A549 Cells with the Compound Represented by Formula I

(1) A549 cells in the logarithmic phase were collected, the concentration of the cell suspension was adjusted, and 100 uL was added per well. The cells to be tested were plated to a density of 10³-10⁴ cells/well and incubated at 37° C. under 5% CO₂ until the bottom of the wells was covered by a single layer of cells.

(2) Different concentrations of the compound of Formula (I) were added: 0, 10, 10², 10³, 10⁴, and 10⁵ nM/well, with 3 replicate wells set, and incubated at 37° C. under 5% CO₂ for 12 hours.

### II. Detection of A549 Cell Activity by CCK-8 Method

(1) 10 μL CCK8 reagent was added to the above cells in each well and reacted for 2 h.
(2) The incubation was terminated, and the culture medium in the wells was carefully aspirated.
(3) The absorbance of each well was measured using a microplate reader with a wavelength of 450 nm.
(4) The absorbance ratio between the experimental group and the control group was calculated as follows: Cell viability=(Absorbance of the experimental group−Blank control)÷(Absorbance of the control group−Blank control)×100%.

The effect of the compound in the example of the present invention on A549 cell activity was as follows:

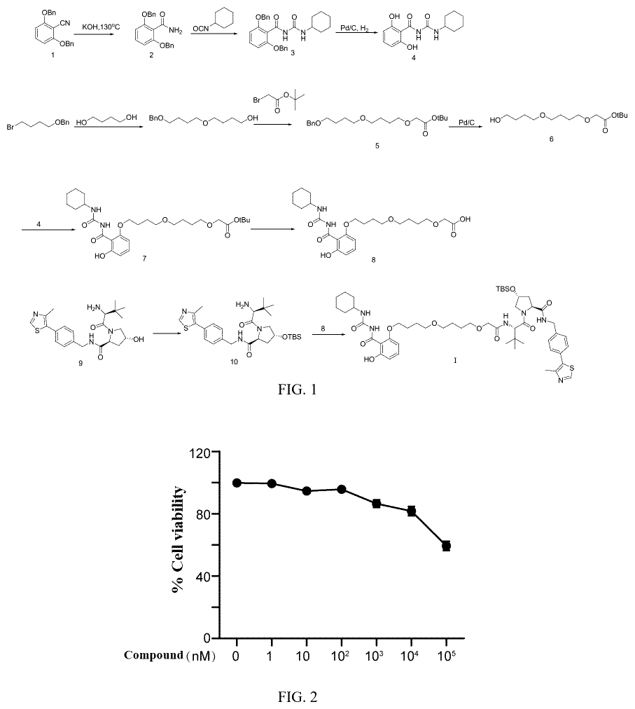

The compound of Formula (I) was less toxic to A549 cells at concentrations ranging from 0 to 10⁵ nM, and the cell viability remained above 80%, as shown in FIG. 2.

### Example 3. Biological Activity Assay of the Compound Represented by Formula I at the Level of Western Blot Experiment

I. Treatment of A549 Cells with the Compound Represented by Formula I

(1) A549 cells in the logarithmic phase were collected, the concentration of the cell suspension was adjusted, and 100 uL was added per well. The cells to be tested were plated to a density of 10³-10⁴ cells/well and incubated at 37° C. under 5% CO₂ until the bottom of the wells was covered by a single layer of cells.

(2) Different concentrations of the compound of Formula I were added: 0, 10, 10², 10³, 10⁴, and 10⁵ nM/well, with 3 replicate wells set, and incubated at 37° C. under 5% CO₂ for 12 hours.

### II. Protein Samples Collection

(1) The treated cells were scraped off from the culture medium, suspended thoroughly, and then collected by centrifugation at 300 g for 5 minutes. After washing with PBS once, the PBS was discarded.

(2) 100 μL of 2×Loading Buffer was added to each sample, shaken thoroughly to mix well, denatured at 100° C. for 5 minutes, and stored at −20° C. after mixing well or directly used for Western Blot detection. The formula of 5×Loading Buffer was as follows: 250 mM Tris-HCl (pH 6.8), 10% (W/V) SDS, 0.5% (W/V) bromophenol blue, 50% (V/V) glycerol, 5% (W/V) β-mercaptoethanol (2-ME). 2×Loading Buffer was prepared by adding 1.5 times the volume of double distilled water to 5×Loading Buffer.

(3) The specific steps for Western Blot (WB) detection were as follows:
1) An appropriate concentration of SDS-PAGE gel was prepared: the concentration of a separating gel of 10% and the concentration of a stacking gel of 5%.
2) Sample preparation: protein samples were prepared according to experimental requirements, centrifuged, mixed well, and loaded into the loading wells of SDS-PAGE gel. The loading volume was adjusted appropriately according to the protein quantification results.
3) Electrophoresis: the power was turned on, and the protein samples were run at 80 volts in the stacking gel. When the protein samples entered the separating gel, the voltage was adjusted to 120 volts to continue the electrophoresis. The electrophoresis was terminated when bromophenol blue almost completely ran out of the PAGE gel.
4) Membrane transfer: after the electrophoresis was completed, the gel was removed, and a membrane transfer device was installed in the following order: (negative electrode), filter paper, gel, activated PVDF membrane, filter paper, (positive electrode). Then, the transfer device was clamped and placed in a membrane transfer buffer, and finally placed in an ice box and placed in a 4° C. refrigerator at a constant voltage of 100V for 40 minutes.
5) Blocking: after the membrane transfer was completed, the PVDF membrane was removed, immersed in TBST buffer containing 5% skimmed milk powder, and shaken on a shaker at room temperature for 1 hour.
6) Primary antibody incubation: after the blocking was completed, the membrane was washed 3 times with TBST buffer, and then an appropriately diluted primary antibody was added and incubated at 4° C. overnight. The PVDF membrane was shaken and washed 3 times with TBST buffer for 5 minutes each time.
7) Secondary antibody incubation: the TBST buffer was discarded, and the diluted secondary antibody was added and shaken on a shaker at room temperature for 1 hour. The secondary antibody was discarded, and the PVDF membrane was shaken and washed 3 times with TBST buffer for 5 minutes each time.
8) Exposure: an ECL chromogenic substrate was evenly spread over the PVDF membrane to expose for imaging.

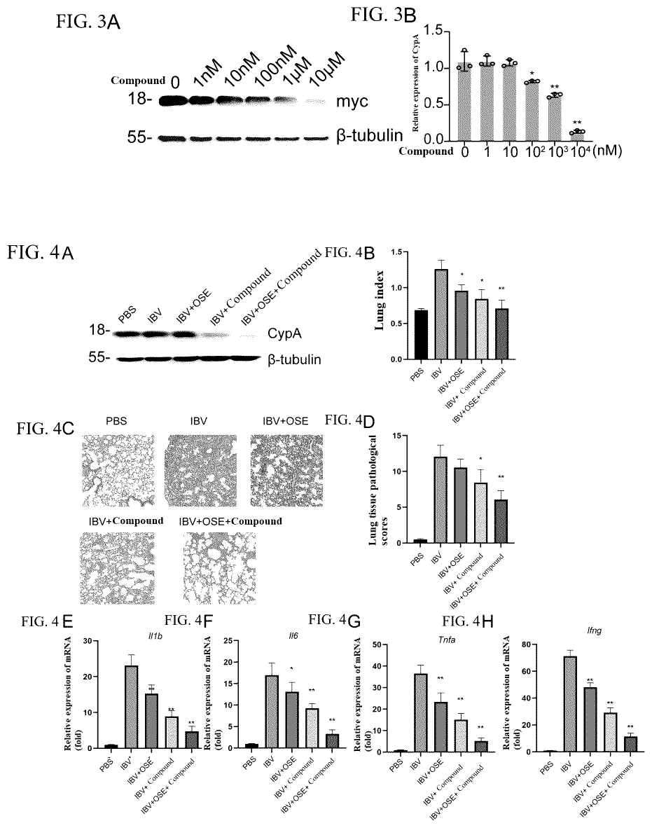

The degradation activity of the compound in the example of the present invention on CypA was as follows: in the A549 cell line, the degradation effect of the compound represented by Formula I on CypA protein could be clearly observed in the WB results, and the half-degradation concentration of the compound on CypA was below 100 nM, as shown in FIGS. 3A and 3B.

### Example 4. Protection Effect of the Compound Represented by Formula I in IBV-Induced Pneumonia

### I. Grouping of Mice and Construction of Mouse Pneumonia Model

Female C57BL/6 mice aged 6-8 weeks were randomly divided into 5 groups: blank group (PBS), untreated group (IBV), treatment group 1 (IBV+OSE (oseltamivir, an anti-influenza virus drug)), treatment group 2 (IBV+compound), and treatment group 3 (IBV+compound+OSE), with 5 mice in each group and a body weight of 19±3 g. C57 BL/6 mice were anesthetized by intraperitoneal injection of Avertin at 200 μL/10 g of body weight. After the mice were anesthetized, 50 μL of PBS was dripped into the nasal cavity of mice in the blank group, and 50 μL of IBV-/Guangxi-Jiangzhou/1352/2018 (8000 PFU) resuspended in PBS was dripped into the nasal cavity of mice in the untreated group, treatment group 1, treatment group 2, and treatment group 3. The mice in treatment group 1 were injected with the compound (10 mg/kg of body weight/day, dissolved in 100 μL of PBS) through the tail vein 24 h after IBV infection. The mice in treatment group 2 were intraperitoneally injected with 0.2 mg of OSE (dissolved in 200 μL of normal saline) at a dose of 10 mg/kg 24 h after IBV infection, and 0.2 mg of OSE was injected once every 24 h. The mice in treatment group 3 were intraperitoneally injected with the compound (10 mg/kg of body weight/day, dissolved in 100 μL of PBS) and 0.2 mg of OSE (dissolved in 200 μL of normal saline) at a dose of 10 mg/kg 24 h after IBV infection, and 0.2 mg of OSE was injected once every 24 h. The blank and untreated groups were given the same dose of normal saline according to the injection time of treatment group 2. The mice in each group were sacrificed 7 days after IBV infection, and subsequent detection experiments were performed.

### II. Detection of CypA Expression in Lung Tissue

(1) Approximately 100 mg of lung tissue from mice in each group was taken, placed in a lysis buffer, and lysed in a tissue crusher at a rotational speed of 4000 rpm for 40 s, with an interval of 10 s, and the process was run twice.

(2) After the run was completed, the product was centrifuged at 12000 rpm at 4° C. for 15 min. The supernatant was taken, added with 5 x loading buffer, and incubated in a metal bath for 10 min for detecting CypA expression by WB experiment.

(3) The WB experiment method was the same as in Example 3.

### III. Lung Index Detection

The mice were anesthetized with ether and weighed. The blood of the mice was collected completely, and the mice were fixed in a supine position. The chest cavity was opened, the esophagus and heart were removed, and the lung tissue was isolated and weighed. The lung index was calculated by weighing the lung of mice: Lung index=Mouse lung weight÷Mouse body weight. The lung index is a very important indicator in determining the severity of pulmonary inflammation, and inflammation will lead to an increase in the lung index.

### IV. Pathological Observation of Mouse Lung Tissue:

(1) The lung tissue of mice was fixed with 4% paraformaldehyde for more than 16 hours, followed by conventional dehydration, wax immersion, embedding, sectioning, and conventional HE staining.

(2) The sample was scored separately according to five indicators: pulmonary interstitial edema, alveolar edema, inflammatory cell infiltration, alveolar hemorrhage, and hyaline membrane formation, as follows: 0 for none; 1 for mild; 2 for moderate; 3 for extensive, and the cumulative sum of the scores was the tissue score.

### V. Detection of Cytokine Expression in Lung Tissue

(1) Approximately 100 mg of lung tissue from mice in each group was taken, placed in 1 mL of Trizol, and lysed in a tissue crusher at a rotational speed of 4000 rpm for 40 s, with an interval of 10 s, and the process was run twice.

(2) After the run was completed, the total mRNA was extracted by the TRIZOL method and reverse transcribed to obtain cDNA for qPCR detection.

(3) qPCR Reaction Procedure:

\textcircled{1} 95° C. for 30 s; \textcircled{2} 95° C. for 30 s; \textcircled{3} 60° C. for 60 s; \textcircled{2} and \textcircled{3} were repreated for 40 cycles.

The fold change was calculated using the 2^{-ΔΔCT} method.

### VI. Experimental Results

The experimental results are shown in FIGS. 4A-4H, where FIG. 4A shows the detection of the expression of CypA in mice of each group, FIG. 4B shows the lung index of mice in each group, FIG. 4C shows the pathology of mice in each group, FIG. 4D shows the pathological scores of mice in each group, FIG. 4E shows the relative expression of Illb mRNA in mice of each group, FIG. 4F shows the relative expression of Il6 mRNA in mice of each group, FIG. 4G shows the relative expression of Tnfa mRNA in mice of each group, and FIG. 4H shows the relative expression of Ifng mRNA in mice of each group.

(1) As can be seen from FIG. 4A, the compound represented by Formula I could significantly reduce the expression of CypA in mouse lung tissue.

(2) As can be seen FIG. 4B, FIG. 4C, and FIG. 4D, the IBV+compound group and the IBV+OSE+compound group could significantly alleviate lung injury in mice. The IBV+OSE+compound group had a better effect.

(3) As can be seen from FIG. 4E, FIG. 4F, FIG. 4G, and FIG. 4H, the IBV+compound group and the IBV+OSE+compound group could significantly reduce the expression of IL-1β, IL-6, TNF-α, and IFN-γ in the lung tissue of mice, thereby alleviating lung inflammation in mice. The IBV+OSE+compound group had a better effect.

### Example 5. Protection Effect of the Compound of Formula (I) in Rheumatoid Arthritis

### I. Grouping of Rats and Construction of Rat Arthritis Model

The BCG vaccine was inactivated in a water bath at 80° C. for 1 h, ground thoroughly with incomplete Freund adjuvant, and mixed well to prepare 15 mg/mL Freund complete adjuvant (FCA). Lewise rats were divided into 4 groups: blank group (PBS), untreated group (UT), treatment group 1 (compound), and treatment group 2 (MTX (methotrexate)), with 5 rats in each group and a body weight of 19±3 g. Except for the PBS group, rats were intradermally injected with 0.1 mL of FCA in the right hind paw. On day 14 after injection, rats in treatment group 1 (compound) were injected with the compound (10 mg/kg of body weight, dissolved in 100 μL of PBS) through the tail vein three times/week: rats in treatment group 2 (MTX (methotrexate)) were intraperitoneally injected with methotrexate (1.2 mg/kg of body weight) three times/week.

### II. Joint Swelling Rate

The circumference of the metatarsophalangeal joint was measured. The circumference of the ankle joint of the left hind paw (on the secondary lesion side) of all rats was measured once before modeling, before administration, and 3 weeks after administration, respectively, and the percentage of joint swelling was calculated.

### III. Joint Index

The joint index was determined by joint swelling score before administration (Day 0) and 3 weeks after administration. Scoring was carried out based on the redness and swelling degree of the ankle and toe joints of the front and rear limbs of rats and the affected joint index, as follows: 0 for normal; 1 for the redness and swelling of one joint; 2 for the redness and swelling of two or more joints; 3 for severe redness and swelling of the paw below the ankle joint; 4 for the redness and swelling of the entire paw including the ankle joint and inability to bear weight. The limbs were scored separately; and the cumulative sum of the scores was the joint index.

### IV. Pathological Examination of Joint Synovium

(1) On the day 21 of administration, rats were euthanized after blood collection. The left metatarsophalangeal joint was fixed with 4% paraformaldehyde for more than 16 hours, followed by decalcification with 10% EDTA, conventional dehydration, wax immersion, embedding, sectioning, and conventional HE staining.

(2) Joint synovial proliferation and the degree of sub-synovial inflammation were observed under a light microscope, and synovial cell proliferation was scored. The synovial cells of normal synovial tissue were monolayer, arranged well, and had a flat shape, with a small amount of inflammatory cell infiltration, without vascular proliferation, fibrosis, or papillary proliferation (scored as 0). In cases of synovial cell proliferation, the number of cell layers slightly increased, fibrous tissue proliferation occurred in the synovial tissue, the capillaries increased, dilated and congested, and inflammatory cell and fibroblast infiltration could be seen (graded and scored from 1-3 depending on the severity).

### V. Experimental Results

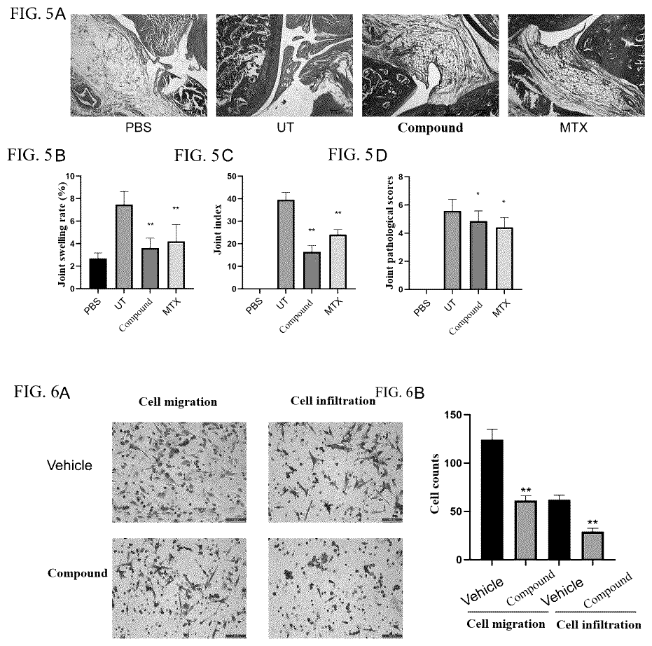

As shown in FIGS. 5A-5D, where FIG. 5A shows the HE images of the ankles of rats in each group, FIG. 5B shows the joint swelling rate of rats in each group, FIG. 5C shows the joint index of rats in each group, and FIG. 5D shows the ankle pathological scores of rats in each group.

As can be seen from FIG. 5A-5D, the compound represented by Formula I in the present invention could significantly relieve joint swelling in rats, reduce the joint index of the ankle joint, and alleviate the inflammation of the ankle joint.

### Example 6. Inhibition of the Compound Represented by Formula I on the Migration and Infiltration of Lung Cancer Cells

### I. Cell Treatment

(1) A549 cells in the logarithmic phase were collected, the concentration of the cell suspension was adjusted, and 100 uL was added per well. The cells to be tested were plated to a density of 10³ to 10⁴ cells/well and incubated at 37° C. under 5% CO₂ until the bottom of the wells was covered by a single layer of cells.

(2) Different concentrations of the compound represented by Formula I were added: 0, 10, 10², 10³, 10⁴, and 10⁵ nM/well, with 3 replicate wells set, and incubated at 37° C. under 5% CO₂ for 12 hours.

The upper chamber of a 24-well cell culture insert (8 μmol/L pore size) was coated (no migration) or (invasive) with a matrix (1 mg/mL) and dried at 37° C. for 30 min.

(3) A549/WT and A549/CypA-cells were trypsinized, washed, and suspended in serum-free DMEM after treatment with 20 ng/ml TGF-β for 48 h.

### II. Determination by Transwell Chambers (Corning Inc.)

(1) DMEM containing 10% fetal bovine serum was added to the lower chamber per approximately 5×10⁴ cells inserted in the upper chamber.

(2) After incubation for 6 h (migration) or 24 h (invasion), non-migrating or invasive cells residing on the upper part of the insert membrane were removed with a cotton swab.

(3) The cells that migrated to the underside of the membrane were fixed and stained with 2% crystal violet in ethanol.

(4) The stained cells were observed and counted under a Japan Olympus CKX41 microscope.

### III. Experimental Results

As shown in FIGS. 6A and 6B, where FIG. 6A shows the migration and infiltration of cells in each group by the Traswell method, and FIG. 6B shows the cell counts of FIG. 6A in FIGS. 6A and 6B.

As can be seen from FIGS. 6A and 6B, the compound represented by Formula I in the present invention could significantly reduce the migration and infiltration of lung cancer cells.

The present invention further comprises the following preferred embodiments:
1. Use of the compound represented by Formula I as described herein for preventing and/or treating CypA-mediated diseases.
2. Use of the compound represented by Formula I as described herein for preventing and/or treating CypA-mediated inflammation, autoimmune diseases, and/or tumors.
3. Use of the compound represented by Formula I as described herein for preventing and/or treating CypA-mediated pneumonia.
4. Use of the compound represented by Formula I as described herein for preventing and/or treating CypA-mediated rheumatoid arthritis, systemic lupus erythematosus, and psoriasis.
5. Use of the compound represented by Formula I as described herein for preventing and/or treating CypA-mediated lung tumors.
6. Use of the compound represented by Formula I as described herein for degrading CypA protein, alleviating influenza virus-induced pneumonia, treating rheumatoid arthritis, and inhibiting the migration and infiltration of cancer cells.
7. The compound of Formula I as described herein for treating CypA-mediated diseases.
8. The compound of Formula I according to embodiment 7, wherein the CypA-mediated diseases are CypA-mediated inflammation, preferably CypA-mediated pneumonia and CypA-mediated rheumatoid arthritis.
9. The compound of Formula I as described herein for treating tumors.
10. The compound of Formula I according to embodiment 9, wherein the tumors are lung tumors.
11. The compound of Formula I as described herein for treating autoimmune diseases.
12. The compound of Formula I according to embodiment 11, wherein the autoimmune diseases comprise systemic lupus erythematosus and psoriasis.
13. A method for treating CypA-mediated diseases in a subject, the method comprising administering to the subject a therapeutically effective amount of the compound of Formula I as described herein.
14. A method for treating tumors in a subject, the method comprising administering to the subject a therapeutically effective amount of the compound of Formula I as described herein.
15. A method for treating autoimmune diseases in a subject, the method comprising administering to the subject a therapeutically effective amount of the compound of Formula I as described herein.

### Industrial Applications

The PROTAC compound provided by the present invention can degrade CypA protein in a targeted manner, so that the PROTAC compound can be used for preparing medicaments for treating inflammation, autoimmune diseases, tumors, and other related diseases. The compound represented by Formula I in the present invention has a significant inhibitory function on virus-induced pneumonia. rheumatoid arthritis. and lung cancer cell migration and infiltration.

1. A compound represented by Formula I; 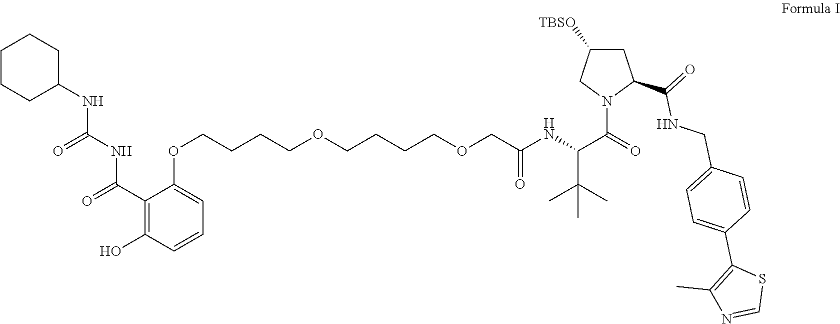
in Formula I, TBSO represents tert-butyldimethylsiloxy.

2. A preparation method of the compound represented by Formula I according to claim 1, comprising the steps of:
S1, subjecting a compound represented by Formula 1 to a hydrolysis reaction in the presence of a basic compound I and benzyl alcohol to obtain a compound represented by Formula 2; 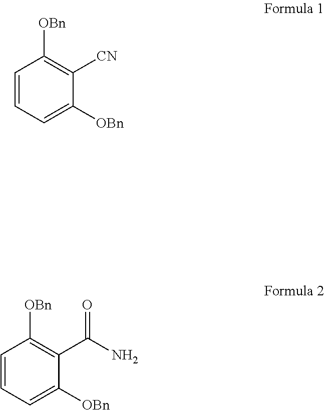
wherein Bn represents benzyl;
S2, subjecting the compound represented by Formula 2 to a nucleophilic addition reaction with isocyanatocyclohexane to obtain a compound represented by Formula 3; 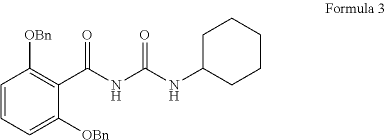 [SMILES: O=C(NC(=O)c1c(OCc2ccccc2)cccc1OCc1ccccc1)NC1CCCCC1]
S3, reducing the compound represented by Formula 3 with palladium on carbon to obtain a compound represented by Formula 4; 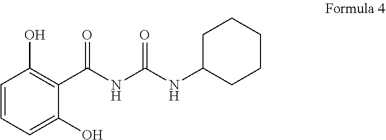 [SMILES: O=C(NC(=O)c1c(O)cccc1O)NC1CCCCC1]
S4, subjecting ((4-bromobutoxy)methyl)benzene to a nucleophilic substitution reaction with butane-1,4-diol in the presence of potassium hydroxide to obtain 4-(4-(benzyloxy)butoxy)butan-1-ol;
S5, subjecting the 4-(4-(benzyloxy)butoxy)butan-1-ol to a nucleophilic substitution reaction with tert-butyl 2-bromoacetate in the presence of tetrabutylammonium bromide and a basic compound II to obtain a compound represented by Formula 5; 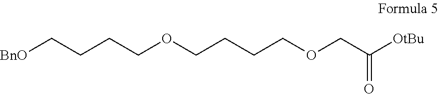 [SMILES: CC(C)(C)OC(=O)COCCCCOCCCCOCc1ccccc1]
in Formula 5, Bn represents benzyl, and tBU represents tert-butyl;
S6, reducing the compound represented by Formula 5 with palladium on carbon to obtain a compound represented by Formula 6; 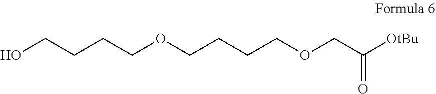 [SMILES: CC(C)(C)OC(=O)COCCCCOCCCCO]
S7, subjecting the compound represented by Formula 6 to a Mitsunobu reaction with the compound represented by Formula 4 in the presence of triphenylphosphine and diisopropyl azodicarboxylate to obtain a compound represented by Formula 7; 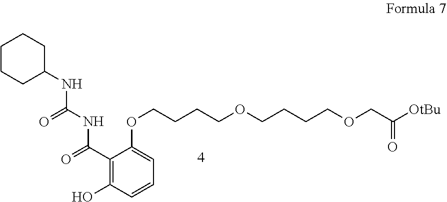 [SMILES: CC(C)(C)OC(=O)COCCCCOCCCCOc1cccc(O)c1C(=O)NC(=O)NC1CCCCC1]
S8, subjecting the compound represented by Formula 7 to a deprotection reaction in the presence of trifluoroacetic acid to obtain a compound represented by Formula 8; 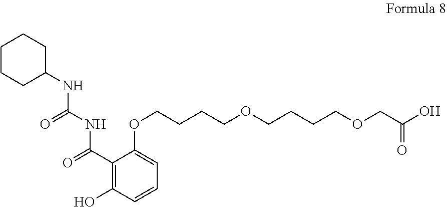 [SMILES: O=C(O)COCCCCOCCCCOc1cccc(O)c1C(=O)NC(=O)NC1CCCCC1]
S9, subjecting a compound represented by Formula 9 to a nucleophilic substitution reaction with tert-butyldimethylsilyl chloride in the presence of N,N-diisopropylethyl amine to obtain a compound represented by Formula 10; 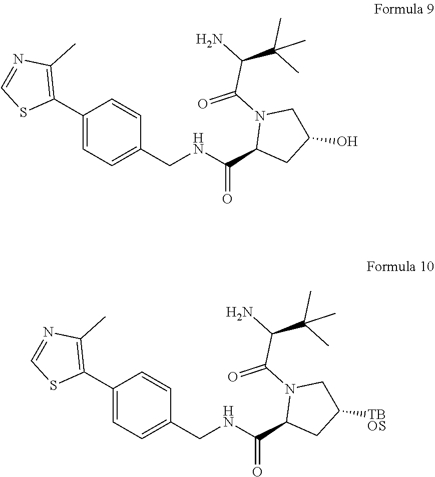
in Formula 10, TBSO represents tert-butyldimethylsiloxy; and
S10, subjecting the compound represented by Formula 8 to a condensation reaction with the compound represented by Formula 10 in the presence of 1-ethyl-(3-dimethylaminopropyl)carbodiimide hydrochloride, 1-hydroxybenzotriazole, and N,N-diisopropylethyl amine to obtain the compound represented by Formula I according to claim 1.

3. The preparation method according to claim 2, wherein in step S1, the basic compound I is KOH;
the molar ratio of the compound represented by Formula 1 to the benzyl alcohol is 1:(5 to 15);
the temperature of the hydrolysis reaction is 120 to 150° C., and the time of the hydrolysis reaction is 8 to 16 h;
the hydrolysis reaction is carried out in water;
in step S2, the nucleophilic addition reaction is carried out under reflux for 12 to 24 h.

4. The preparation method according to claim 2, wherein in step S4, the molar ratio of the ((4-bromobutoxy)methyl)benzene to the butane-1,4-diol is 1:(2 to 5);
the temperature of the nucleophilic substitution reaction is 20 to 40° C., and the time of the nucleophilic substitution reaction is 1 to 5 h;
in step S5, the basic compound II is NaOH;
the molar ratio of the 4-(4-(benzyloxy)butoxy)butan-1-ol to the tert-butyl 2-bromoacetate is 1:(1 to 5);
the temperature of the nucleophilic substitution reaction is 20 to 40° C., and the time of the nucleophilic substitution reaction is 12 to 24 h.

5. The preparation method according to claim 4, wherein in step S7, the molar ratio of the compound represented by Formula 6 to the compound represented by Formula 4 is 1:(1 to 1.5);
the molar ratio of the compound represented by Formula 6, the triphenylphosphine, and the diisopropyl azodicarboxylate is 1:(1 to 2):(1 to 2);
the temperature of the condensation reaction is 0 to 25° C., and the time of the condensation reaction is 1 to 5 h;
in step S8, the temperature of the deprotection reaction is 20 to 40° C., and the time of the deprotection reaction is 1 to 5 h;
in step S9, the molar ratio of the compound represented by Formula 9 to tert-butyldimethylsilyl chloride is 1:(1 to 5);
the molar ratio of the compound represented by Formula 9 to the N,N-diisopropylethyl amine is 1:(5 to 15);
the temperature of the substitution reaction is 0 to 25° C., and the time of the substitution reaction is 12 to 18 h;
in step S10, the molar ratio of the compound represented by Formula 8 to the compound represented by Formula 10 is 1:(1 to 1.5);
the molar ratio of the compound represented by Formula 8, the 1-ethyl-(3-dimethylaminopropyl)carbodiimide hydrochloride, the 1-hydroxybenzotriazole, and the N,N-diisopropylethyl amine is 1:(1 to 2):(1 to 2):(1 to 2);
the temperature of the condensation reaction is 0 to 25° C., and the time of the condensation reaction is 12 to 18 h.

6. Use of the compound represented by Formula I according to claim 1 in the preparation of medicaments for preventing and/or treating CypA-mediated diseases.

7. The use according to claim 6, wherein the CypA-mediated diseases comprise CypA-mediated inflammation, CypA-mediated autoimmune diseases, and/or tumors.

8. The use according to claim 7, wherein the inflammation comprises pneumonia;
the autoimmune diseases comprise rheumatoid arthritis, systemic lupus erythematosus, and psoriasis;
the tumors comprise lung tumors.

9. Use of the compound represented by Formula I according to claim 1 for preparing a product having any of the following functions;
1) degrading CypA protein;
2) alleviating influenza virus-induced pneumonia;
3) treating rheumatoid arthritis;
4) inhibiting the migration and infiltration of cancer cells.

10. A pharmaceutical composition comprising the compound represented by Formula I according to claim 1 as an active ingredient and at least one pharmaceutically acceptable carrier, excipient, and/or diluent.

11. A method for treating CypA-mediated diseases in a subject, the method comprising administering to the subject a therapeutically effective amount of the compound represented by Formula I according to claim 1.

12. The method according to claim 11, wherein the CypA-mediated diseases comprise inflammation, autoimmune diseases, and/or tumors.

13. The method according to claim 12, wherein the inflammation comprises pneumonia;
the autoimmune diseases comprise rheumatoid arthritis, systemic lupus erythematosus, and psoriasis;
the tumors comprise lung tumors.

14. The compound represented by Formula I according to claim 1 for treating CypA-mediated diseases.

15. The compound represented by Formula I according to claim 14, wherein the CypA-mediated diseases comprise inflammation, autoimmune diseases, and/or tumors.

16. The compound represented by Formula I according to claim 15, wherein the inflammation comprises pneumonia;
the autoimmune diseases comprise rheumatoid arthritis, systemic lupus erythematosus, and psoriasis;
the tumors comprise lung tumors.
